(19) United States
(12) Patent Application Publication
(10) Pub. No.: US 2026/0191663 A1
(43) Pub. Date: Jul. 9, 2026

(54) CRYOPRESERVED OSTEOCHONDRAL ALLOGRAFT (COCA) TRANSPLANTATION DEFECT SIZERS AND INSTRUMENT KITS

(71) Applicant: Allosource, Centennial, CO (US)

(72) Inventors: Adam Yanke, Chicago, IL (US); Jordyn Hammer, Elizabeth, CO (US); Lauren Castillo, Castle Rock, CO (US); Katie Kane, Highlands Ranch, CO (US); Reginald Stilwell, Parker, CO (US)

(21) Appl. No.: 19/440,083

(22) Filed: Jan. 5, 2026

Related U.S. Application Data
(60) Provisional application No. 63/741,803, filed on Jan. 3, 2025.

(51) Int. Cl. A61F 2/46; A61F 2/28; A61L 27/36

(52) U.S. Cl. CPC A61F 2/4601; A61L 27/3608; A61L 27/3612; A61F 2002/2839; A61F 2002/4659; A61F 2310/00359; A61L 2430/02; A61L 2430/06

(57) ABSTRACT

There is disclosed a defect sizer tool for selection of an appropriately sized pre-cut core for a cryopreserved osteochondral allograft (COCA) transplantation procedure. In an embodiment, the tool includes an elongated connector that provides a handle portion. A ring measurement guide at one end, with a given diameter, and alignable with a cartilage defect to provide an estimated measurement of a diameter. There is disclosed an instrument kit with a pin, a pin guide, and a reamer configured to slidingly receive the pin, blades at one end, and a rotational drive connector at the other end. In an embodiment, a method for a COCA transplantation procedure includes providing an instrument kit, using the pin guide to place the pin in the subchondral bone, and operating the reamer to score surrounding cartilage prior to the reamer bit portion reaming the subchondral bone to the desired depth. Other embodiments are also disclosed.

### REFERENCE TO PENDING PRIOR PATENT APPLICATION

This application claims the benefit under 35 U.S.C. 119 (e) of U.S. Provisional Patent Application No. 63/741,803, filed Jan. 3, 2025 by Adam Yanke, et al., for “CRYOPRESERVED OSTEOCHONDRAL ALLOGRAFT (COCA) TRANSPLANTATION DEFECT SIZERS AND INSTRUMENT KITS” which patent application is hereby incorporated herein by reference.

### BACKGROUND

Trauma and/or disease to articulating joints (e.g., the knee joint) can cause structural damage to the articular cartilage surface and the underlying subchondral bone, resulting in pain and/or disability for the patient. The area of the articular cartilage (and/or underlying subchondral bone) that is damaged is commonly referred to as a “lesion”. Lesions may develop as the result of disease that affects the cartilage and/or bone or, perhaps more commonly, as the result of sporting injuries in which shear forces cause a separation of cartilage between the radial and calcified layers of bone. Chondral lesions lie entirely within the cartilage and do not penetrate into the subchondral bone. Osteochondral lesions penetrate the articular surface in depth, into the vascularized subchondral bone.

To treat an osteochondral lesion, a surgeon must remove the diseased/damaged cartilage together with a portion of the underlying bone. The resulting cavity is then filled with either a cellular graft, an osteochondral graft, or an allograft/autograft derived cartilage implant. For the purposes of the instant disclosure, the term “allograft” is intended to mean a donor derived or artificially derived (e.g., synthetic, lab-grown, etc.) tissue graft, and the term “autograft” is intended to mean a tissue graft obtained from the patient that is being treated (i.e., a tissue graft transferred from one area of the patient's body to the treatment site).

Currently, surgeons perform osteochondral restoration using a variety of different surgical instruments, often in concert with one another. By way of example, a surgical coring tool comprising a cylindrical thin-walled tube enclosing a lumen having a fixed cutting blade disposed perpendicular to the axis of the lumen may be used, with the surgeon manually rotating the coring tool in order to “core out” a plug of tissue and bone. Alternatively, a cannulated surgical drill bit configured to fit over a guidewire may be used in concert with an appropriate surgical drill in order to “drill out” a plug of tissue and bone. Alternatively, a ring curette and bone curette may be used to outline the lesion and another, smaller curette may be used to “remove” a plug of material outlined by the ring or bone curette.

It will be appreciated that each of these surgical methods and apparatus result in a time-consuming procedure requiring a variety of different tools which sometimes do not match the size/geometry of the lesion that is to be removed. Moreover, tools such as a manually-operated coring tool, a drill bit or a curette require significant skill on the part of the surgeon in order to remove the lesion and prepare a cavity of the proper depth and size to receive the graft.

Thus, there is a need for a new and improved method and apparatus for performing osteochondral restoration that combines the functions of various surgical instruments into a single device, which permits the surgeon to precisely locate and core circular sections of cartilage and bone to a precise depth so as to create a cavity for receiving a graft.

### SUMMARY

This Summary is provided to introduce a selection of concepts in a simplified form that are further described below in the Detailed Description. This Summary is not intended to identify key aspects or essential aspects of the claimed subject matter. Moreover, this Summary is not intended for use as an aid in determining the scope of the claimed subject matter.

In an embodiment, there is provided a defect sizer tool as a measurement guide tool for selection of an appropriately sized pre-cut core for a COCA transplantation procedure with a corresponding instrument set.

In one embodiment, the defect sizer tool provides a measurement guide tool for selection of an appropriately sized pre-cut core for a cryopreserved osteochondral allograft (COCA) transplantation procedure with a corresponding instrument set. The defect sizer tool includes a first end and a second end in opposition to one another, an elongated connector extending between the first end and the second end, and the elongated connector providing a handle portion configured to allow a user to grasp the handle portion between the first end and the second end. A first ring measurement guide is disposed at the first end, the first ring measurement guide has a first given diameter, and the first ring measurement guide is configured to align with a cartilage defect to provide a visual indication for an estimated measurement of a diameter of the cartilage defect.

In another embodiment, the defect sizer tool further includes a second ring measurement guide disposed at the second end. The second ring measurement guide has a second given diameter. The first given diameter of the first second ring measurement guide and the second given diameter of the second ring measurement guide are unequal to one another so as to provide at least two measurement guides on a single defect sizer tool. The second ring measurement guide is configured to align with a cartilage defect to provide a visual indication for an estimated measurement of a diameter of the cartilage defect.

In yet another embodiment, the first given diameter of the first ring measurement guide is an outer diameter of the first ring measurement guide. The second given diameter of the second ring measurement guide is an outer diameter of the first ring measurement guide. The outer diameter of the first ring measurement guide and the outer diameter of the second ring measurement guide are unequal to one another so as to provide at least two measurement guides on a single defect sizer tool.

In still yet another embodiment, the first ring measurement guide includes a second additional given diameter configured to align with a cartilage defect to provide a visual indication for an estimated measurement of a diameter of the cartilage defect. The second additional given diameter of the first ring measurement guide is an inner diameter of the first ring measurement guide. The second ring measurement guide includes a second additional given diameter configured to align with a cartilage defect to provide a visual indication for an estimated measurement of a diameter of the cartilage defect. The second additional given diameter of the second ring measurement guide is an inner diameter of the second ring measurement guide. The outer diameter of the first ring measurement guide, the second additional given diameter of the first ring measurement guide, the outer diameter of the second ring measurement guide, and the second additional given diameter of the second ring measurement guide are each unequal to one another so as to provide at least four measurement guides on the defect sizer tool.

In another embodiment, there is provided an instrument set (which may be provided as an instrument kit) for a COCA transplantation procedure, the instrument set includes a pin, a pin guide, a reamer with adjustable stop, a depth guide, and a dilator with tamp.

In still another embodiment, there is provided an instrument kit for a cryopreserved osteochondral allograft (COCA) transplantation procedure. The instrument kit includes a pin having a first end and a second end in opposition to one another, a given outer diameter, and the first end configured to penetrate subchondral bone. A pin guide has a first end and a second end, the pin guide forming a passageway extending between the first end and the second end, the passageway configured to slidingly receive the pin, with the width of the passageway corresponding to the given outer diameter of the pin to allow sliding motion of the pin therein without deflection from a longitudinal axis extending between the first end and the second end of the pin. A reamer is provided with an adjustable stop. The reamer forming a passageway extending between the first and the second end. The passageway configured to slidingly receive the pin, with the width of the passageway corresponding to the given outer diameter of the pin to allow sliding motion of the pin therein without deflection from a longitudinal axis extending between the first end and the second end of the pin. A set of blades positioned at the first end. A rotational drive connector positioned at the second end.

In yet another embodiment, there is provided a method for a COCA transplantation procedure, the method including providing and using an instrument set configured for the COCA transplantation procedure with specified steps related to the instrument set configured of the COCA transplantation procedure.

In still another embodiment, a method for a COCA transplantation procedure includes providing an instrument kit, using the pin guide to place the pin in the subchondral bone, and operating the reamer to first score surrounding cartilage prior to the reamer bit portion reaming the subchondral bone to the desired depth.

Other embodiments are also disclosed.

Additional objects, advantages and novel features of the technology will be set forth in part in the description which follows, and in part will become more apparent to those skilled in the art upon examination of the following, or may be learned from practice of the technology.

### BRIEF DESCRIPTION OF THE DRAWINGS

Non-limiting and non-exhaustive embodiments of the present invention, including the preferred embodiment, are described with reference to the following figures, wherein like reference numerals refer to like parts throughout the various views unless otherwise specified. Illustrative embodiments of the invention are illustrated in the drawings, in which:

FIG. 1A illustrates a defect sizer as a measurement guide tool for selection of an appropriately sized pre-cut core for a COCA transplantation procedure with a corresponding instrument set;

FIG. 1B illustrates another view of the defect sizer of FIG. 1A;

FIG. 2 illustrates an instrument set (which may be provided as an instrument kit) for a COCA transplantation procedure, the instrument set includes a pin, a pin guide, a reamer with adjustable stop, a depth guide, and a dilator with tamp (as provided from top to bottom of the view);

FIG. 3 illustrates the pin of FIG. 2;

FIG. 4 illustrates the pin guide of FIG. 2;

FIGS. 5A-5K illustrates the reamer with adjustable stop;

FIG. 6 illustrates the depth guide of FIG. 2;

FIGS. 7A-7B illustrates the dilator with tamp of FIG. 2;

FIGS. 8A-8G illustrate steps of an operative technique for a COCA transplantation procedure using the defect sizer of FIGS. 1A and 1B, and using the instrument set of FIG. 2.

### DETAILED DESCRIPTION

Embodiments are described more fully below in sufficient detail to enable those skilled in the art to practice the system and method. However, embodiments may be implemented in many different forms and should not be construed as being limited to the embodiments set forth herein. The following detailed description is, therefore, not to be taken in a limiting sense.

In various embodiments, single-use instruments and sizer sets may support procedures using cryopreserved pre-cut core allografts for full-thickness cartilage lesions and facilitate press fitting of the cores. The industry is specific to cartilage restoration in the area of orthopedics. Unlike similar instruments on the market, these are not reusable and they also consist of 5 pieces which adds to the specificity of the set compared to other instruments on the market. The instruments are used with a spectrum of products, including, for example, but not limited to, cryopreserved cores available in various sizes, which may include, but are not limited to the following sizes: 11 mm, 16 mm and 20 mm. Currently, the market only offers “fresh” cores that have limited availability due to the “fresh” core shelf-life of 28 days. The instruments may support the new cryopreserved cores that will provide a 2-year shelf life making the cartilage product available to more patients and surgeons.

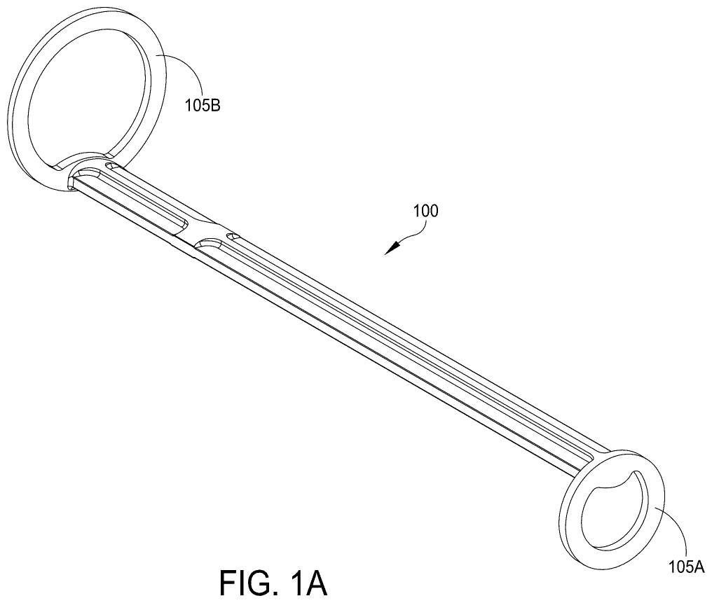

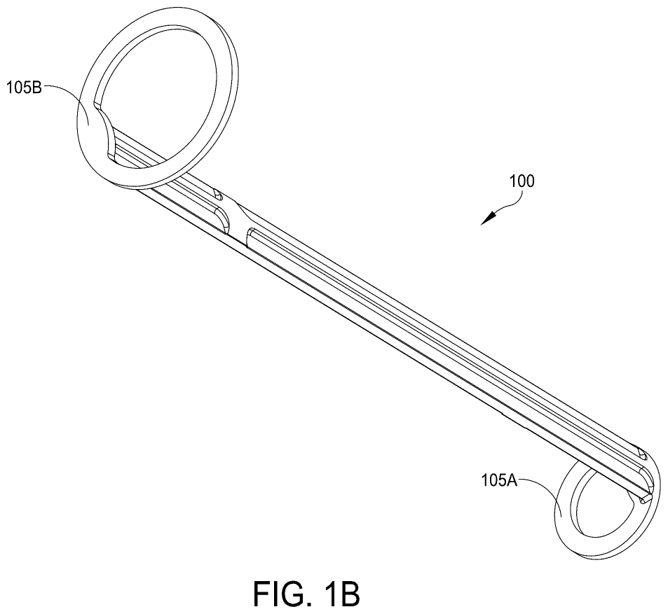

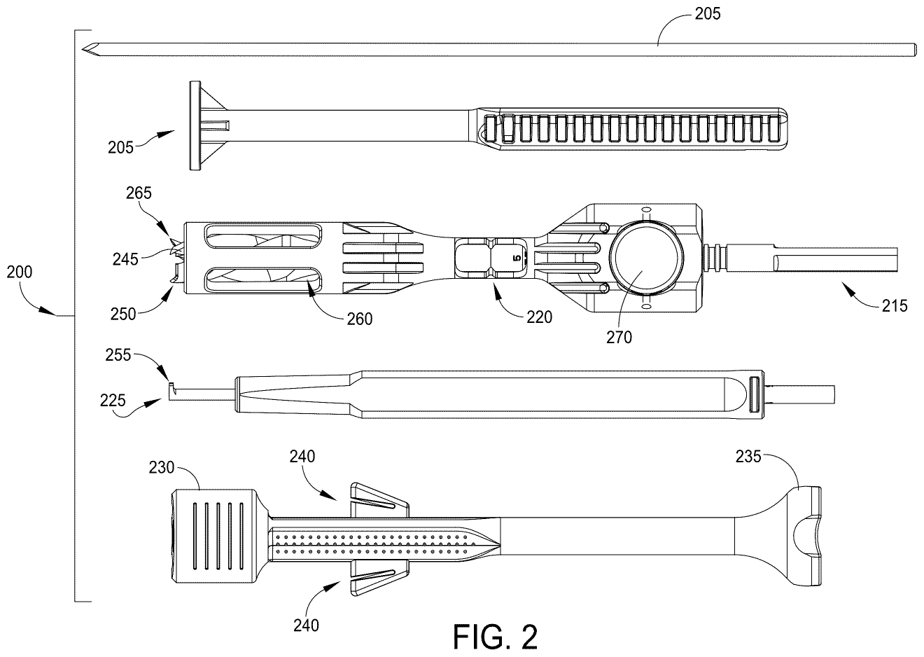

With reference to FIGS. 1A and 1B, there is shown a defect sizer tool 100 which provides a measurement guide 105A, 105B that is used to select the appropriately sized pre-cut core and corresponding instrument set 200 (FIG. 2). In one embodiment, defect sizer has a first ring measurement guide 105A and a second ring measurement guide 105B. First ring measurement guide 105A and second ring measurement guide 105B may be sized larger and smaller than one another on one defect sizer tool 100 or on separate defect sizers. One, two, or more different sized ring measurement guides may be provided on one defect sizer tool or multiple defect sizer tools. In an embodiment, first ring measurement guide 105A may be provided as a small ring 105A and second ring measurement guide 105B may be provided as a large ring 105B that can be aligned with the cartilage defect to estimate the defect diameter. In an embodiment, the smaller ring 105A has an inner diameter of 11 mm and an outer diameter of 16 mm. In an embodiment, the larger ring 105B has an inner diameter of 20 mm and an outer diameter of 25 mm. Other appropriate sizes may be implemented.

In various embodiments, and with reference to FIG. 2, there is shown instrument set 200 which may include a pin 205, a pin guide 210, a reamer 215 with an adjustable stop 220, a depth guide 225, and a dilator 230 with a tamp 235 and suture grips 240.

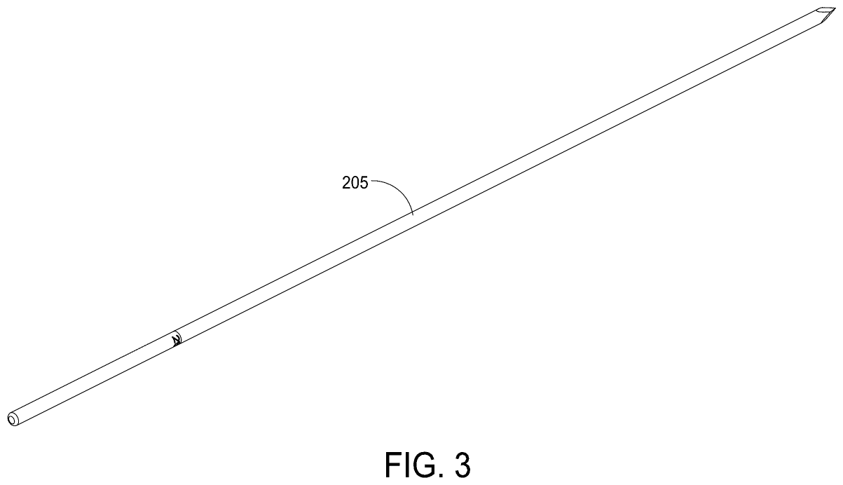

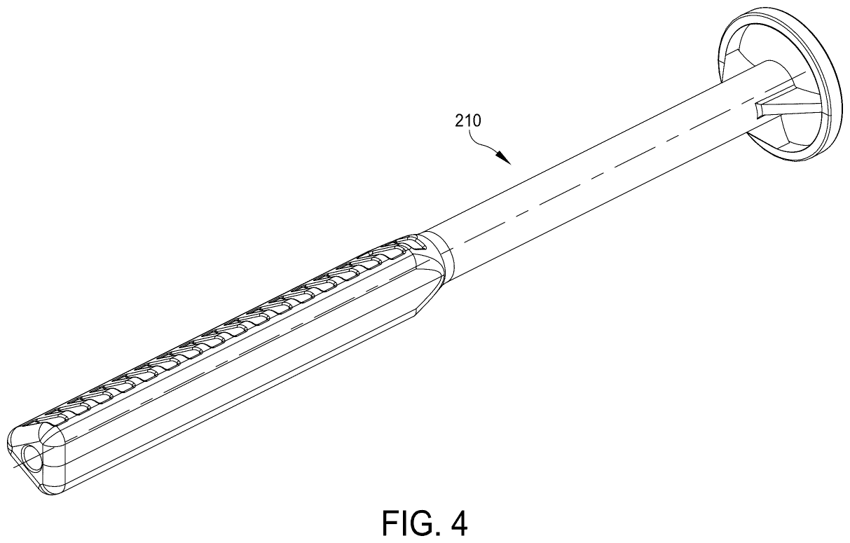

In various embodiments, and with reference to FIGS. 3 and 4, pin 205 (FIG. 3) may be configured with a suitable length for a COCA transplantation procedure. For example, and unlike other orthopedic procedures, pin 205 (FIG. 3) may be configured with a minimal length to extend through the corresponding pin guide 210 (FIG. 4) with a minimal amount of excess configured to emerge from either side of pin guide 210 (FIG. 4).

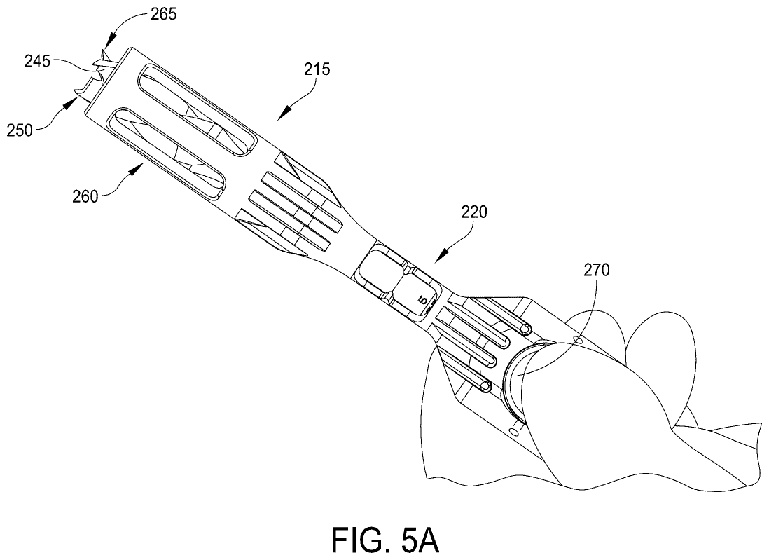

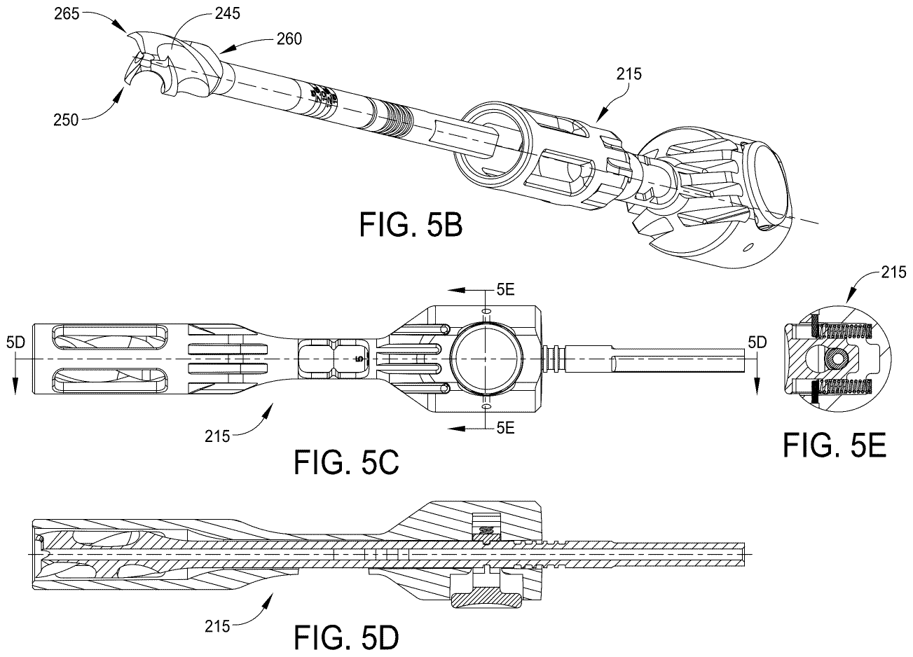

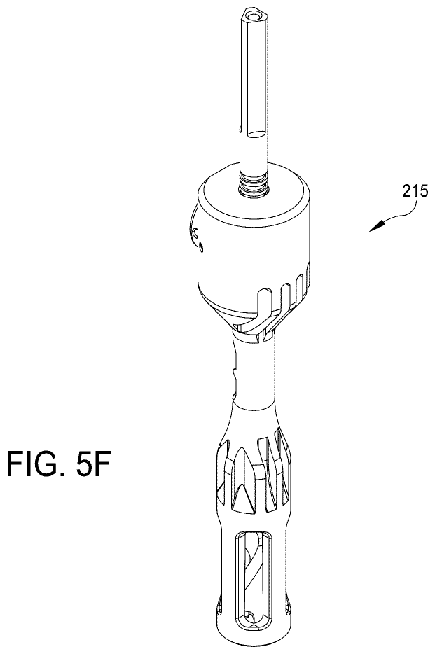

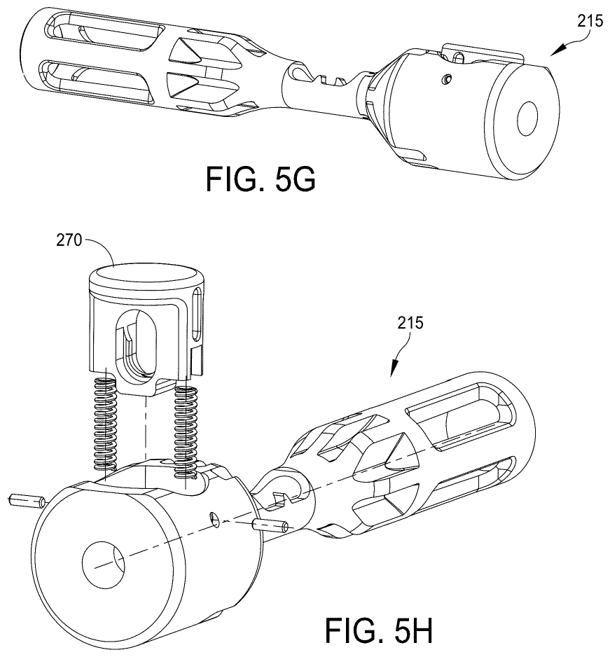

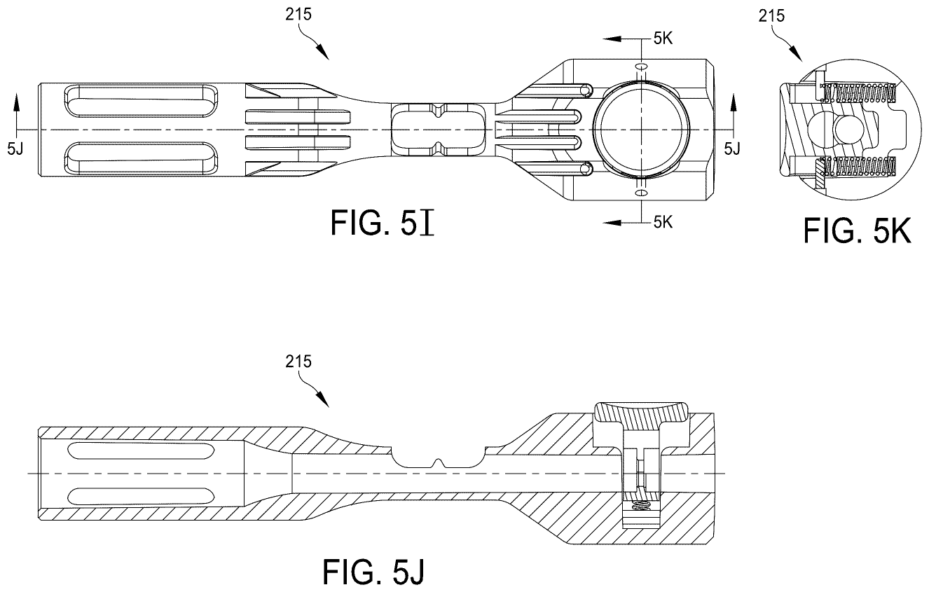

In various embodiments, and with reference to FIGS. 5A-5K, reamer 215 may be configured to provide adjustments depths of 5, 7.5, 10, 12.5 and 15 mm. In one embodiment, reamer 215 has a unique blade construction 245 with winged blades 250 so that reamer 215 can score the cartilage before cutting to get a cleaner cut in the surgical site.

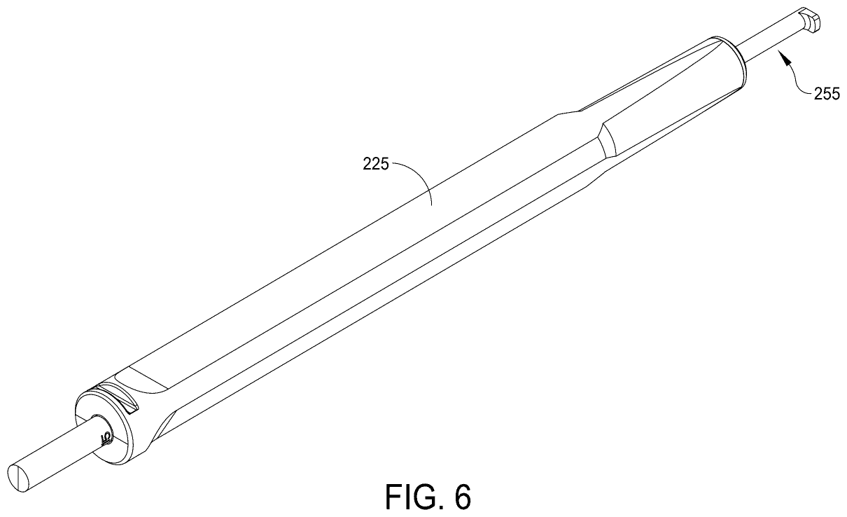

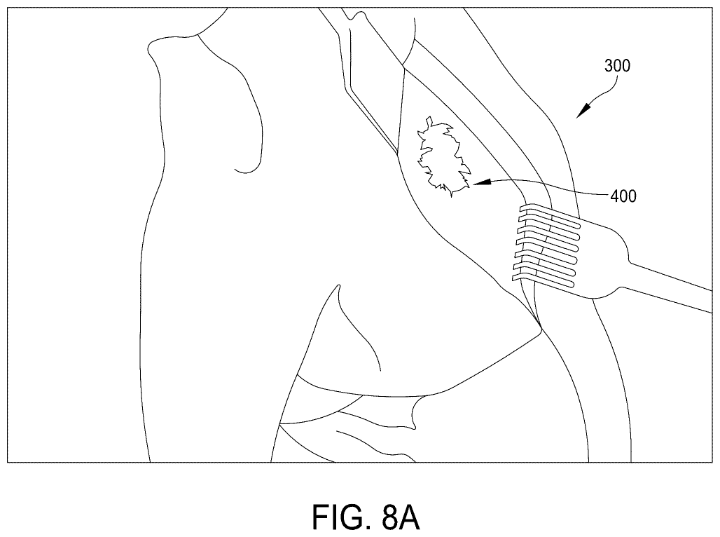

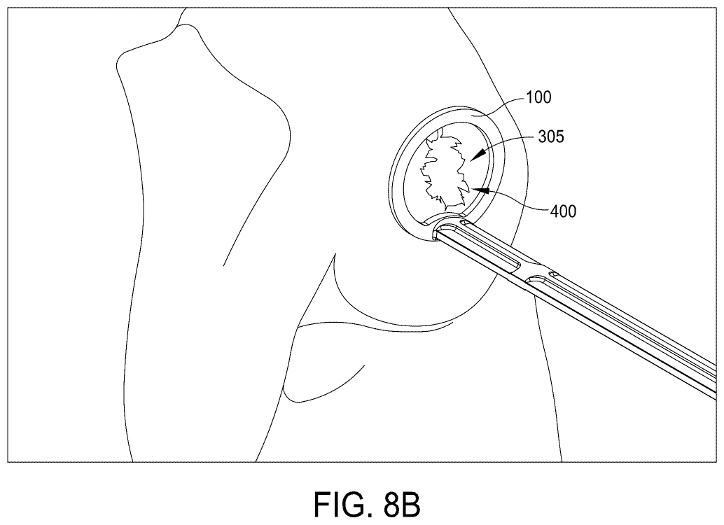

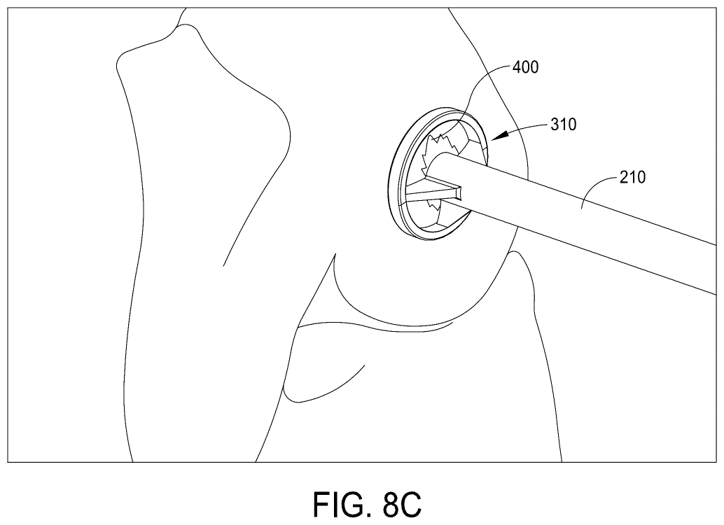

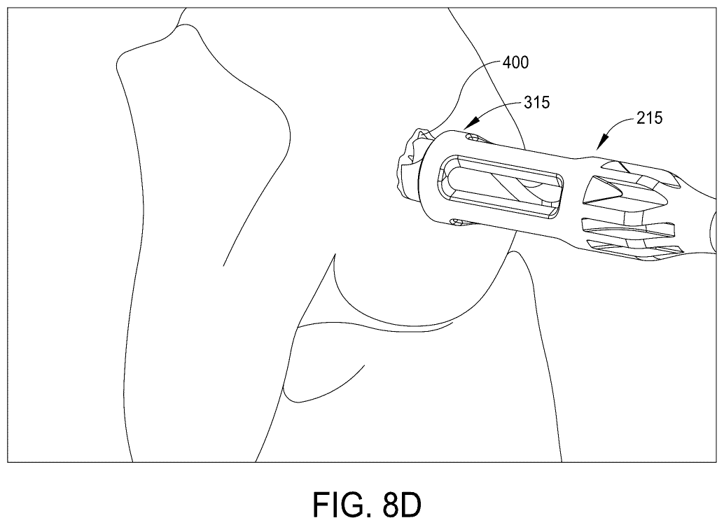

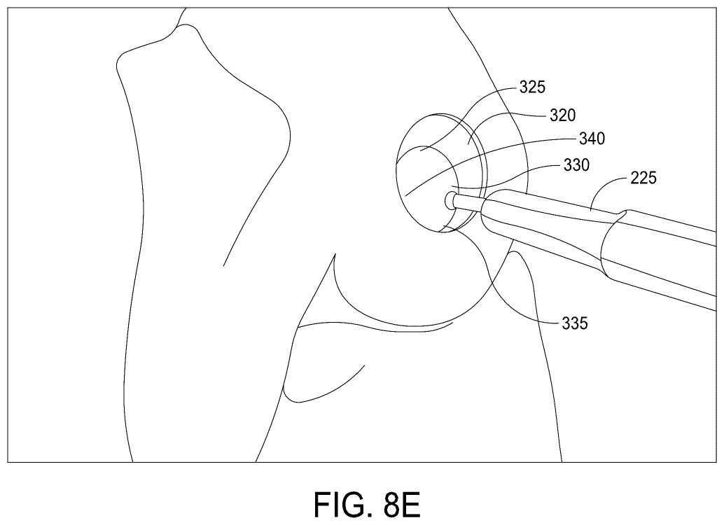

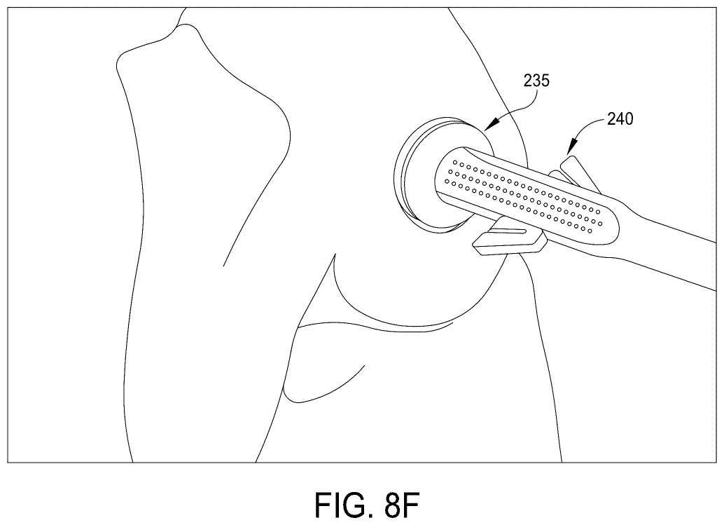

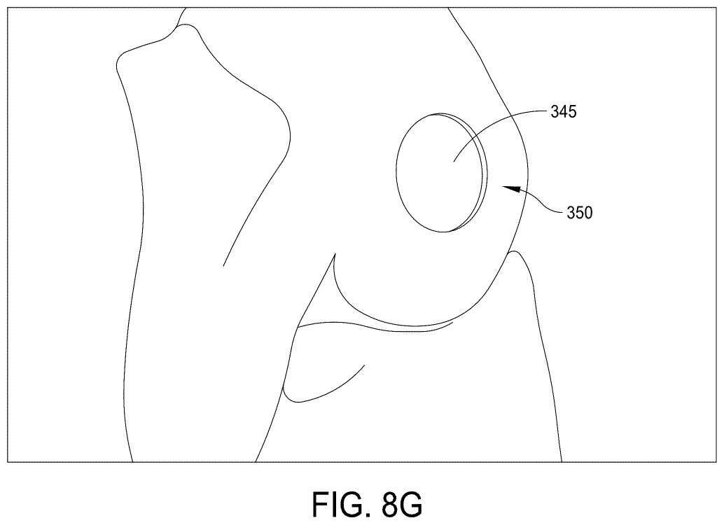

In various embodiments, and with reference to FIG. 6, depth guide 225 may be configured to allow a surgeon to verify the depth of the reamed defect and to ensure that the cut was made perpendicularly. This allows the clinician to effectively place the graft at the 12, 3, 6, and 9 o'clock positions (discussed below with respect to FIG. 8E) based on the depth measured at each of these positions in the surgical sight.

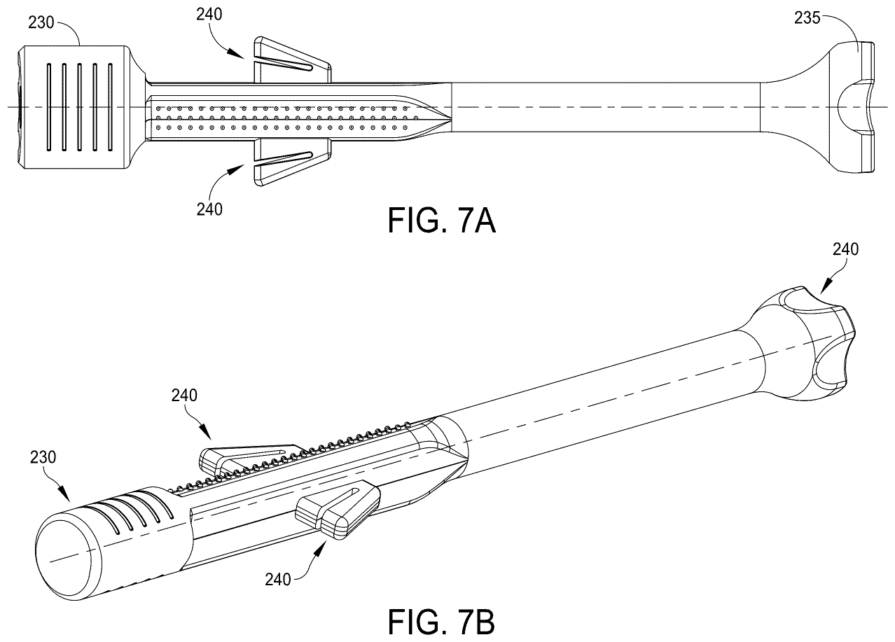

In various embodiments, and with reference to FIGS. 7A and 7B, the dilator 230 with tamp 235 includes not only the dilator 230 but also suture grips 240 that can be used to wrap suture around the distal portion of a graft and remain in selective attachment to the suture grips 240 so as to allow the graft to be easily removed from the patient, if needed. The tamp 235 is contoured with a 20 mm radius of curvature (ROC) to contour to the anatomy of the cores that are being inserted into the surgical site. The concavity of the reamer 215 is also to ensure the integrity of the cartilage, minimizing any damage to the core from contact during insertion.

These specific instruments may be configured for use with cryopreserved cores to ensure that the size of the lesion is properly measured and that the reaming and ability to remove the graft is available should the graft be too tight or too loose. This kit 200 is specifically made to be a sized match for the cores made and validated at a tissue processor, e.g., at AlloSource. The size of the cores and the mismatch created by the reamer have been validated to fit according to surgical expectations.

The instrument development process included challenges in the tolerances of each of the sizes to be sufficient for the actual core size. It has to be loose enough to not fall out yet tight enough for a press-fit, while not being too tight that it caused damage to the surrounding cartilage. In addition, initially the dilator 230 was not part of the instrument set 200, however, as the development proceeded, it was found that dilator 230 ensures the press-fit and accurate placement for the cryopreserved core. The dilator 230 may be configured to size the recipient site to accept an implant. The dilator 230 may include features to inhibit the creation of a vacuum when inserted into the recipient site. The shape of the dilator 230 may be slightly tapered, there may be holes or scored portions or other variances, or a combination of one or more of tapering, holes, and scoring to prevent vacuum formation.

The depth guide 225 may be spring loaded for ease of use to allow a clinician to obtain a more accurate match when inserting the core by pre-measuring the depth of each of the different locations (12, 3, 6, and 9 o'clock). There are currently no tools on the market like this that are provided with coring instrument sets. The current standard device on the market is a ruler which can have inaccuracies due to the anatomy of the reamed site. The right angle 255 of the depth stop 225 allows the clinician to get flush to the reamer 215 and obtain a more precise measurement.

The reamer 215 itself acts as both a reamer bit 260 and scoring bit 265. Some kits have separate scoring bits that are used prior to reaming. This bit acts as both through the teeth 260, 265 that are located on the edge of the blade 245. The scoring bit 265 first scores the surrounding cartilage before the reamer bit 260 begins reaming to the desired depth.

The dilator/tamp combo 230, 235 is unique as it acts as two tools in one. The tamp end 235 of the dilator 230 is contoured with the concavity of a core to ensure there is contact around the entire surface of the core when it is inserted. This ensures direct contact is distributed across the core, minimizing damage and allowing for easier insertion of the core. The tamp/dilator combo 230, 235 also has a special suture grip 240 that allows the clinician to pre-place the core to ensure that it is a good fit (i.e. not proud nor too shallow). This special feature provided by suture grip 240 allows for easy removal of the core. A feature that other tools do not have.

With reference to FIGS. 8A-8G, there may be provided various embodiments incorporating operative techniques for a COCA transplantation procedure using instrument set 200 for a COCA transplantation procedure. These may include, but are not limited to, with one or more of the steps described herein that may be utilized. Initially, defect sizer tool 100 may be provided to determine the size of the cartilage lesion.

### Step 1—FIG. 8A

A standard parapatellar arthrotomy is preformed 300 to expose the cartilage defect 400. Exposure is often improved by placing a retractor in the femoral notch and on the outer aspect of the condyle. Selected fat pad excision may also aid in exposure. Flexion and extension of the knee can be used to expose lesions that are more posterior or anterior, respectively.

### Step 2—FIG. 8B

The defect sizer tool 100 is placed aseptically onto the sterile field 305. The circular diameter end of the Defect Sizer is aligned with the patient's cartilage defect 400, fully encompassing the defect 400 within the defect sizer tool 100.

### Step 3

The Pre-Cut Core of diameter large enough to cover the defect is selected. The Pre-Cut Core vial aseptically placed onto the sterile field prior to thawing. To thaw the Pre-Cut Core, place the unopened vial in a warm or room temperature sterile saline solution. The minimum thaw time can range from 10-30 minutes, but should not exceed one hour. When fully thawed, the Pre-Cut Core is removed from the vial and immediately placed in warm or room temperature sterile saline until ready to implant. The Pre-Cut Core should be completely submerged in the saline. When it is time to implant, remove the Pre-Cut Core from the saline and pulse lavage to adequately remove residual cryoprotectant medium and marrow elements.

In various embodiments, a sterile instrument set is provided for a cryopreserved osteochondral allograft transplantation procedure. That is, the materials of construction must be fully compatible with sterilization methods of choice which will mainly be gamma irradiation. Gama irradiation is typically done at a dosage of 25 kGy in order to obtain a Sterility Assurance Level of 10(−6) according to FDA medical device guidelines. All polymeric and other materials of construction are made from gamma compatible materials. These radiation compatible materials of construction form the Pre-Cut Core since the final “product” may be offered as “sterile” or SAL6. “SAL6” sterility means the absence of all viable microorganisms including viruses. At present, a sterility assurance level (SAL) of 10-6 is generally accepted for pharmacopoeial sterilization procedures, i.e., a probability of not more than one viable microorganism in an amount of one million sterilized items of the final product.

### Step 4—FIG. 8C

The corresponding instrument set (not shown) may be placed aseptically onto the sterile field 310. The pin guide 210 should be placed over the cartilage defect 400, fully encompassing the defect 400, while being centered. The guide 210 should be flush and perpendicular to the cartilage surface. Drill the pin 205 through the center of the pin guide 210 and into the subchondral bone. Remove the pin guide 210 and keep the pin 205 in place.

### Step 5—FIG. 8D

The cutting depth of the reamer 215 with the adjustable stop 220 may be adjusted by depressing the button 270 (FIG. 2) and sliding to the desired depth (e.g., 5 mm, 7.5 mm, 10 mm, 12.5 mm, and 15 mm). To create the recipient socket, attach the reamer 215 to a drill and ream coaxially along the pin 205 into the subchondral bone until the adjustable stop 220 is flush with the cartilage surface 315. The socket should be cleaned out to remove any residual cartilage and bone debris.

### Step 6—FIG. 8E

Depth of socket 320 should be confirmed with the depth guide 225 at the 12 o'clock position 325, the 3 o'clock position 330, the 6 o'clock position 335 and the 9 o'clock position 340.

### Step 7—FIG. 8F

Tamp the dilator 230 into the recipient socket 230 until fully seated and leave it there while preparing the thawed pre-cut core. Next, cut the length of the pre-cut core with a micro sagittal saw, or similar device, to the corresponding depth of the recipient socket 320, measured per the depth guide 225. Chamfer, or round off, the bone edges of the pre-cut core with a small rongeur or rasp to aid in introducing the pre-cut core into the recipient socket 230.

### Step 9—FIG. 8G

Remove the dilator 230 (FIG. 2) and, if needed, gentle tamping using the contoured tamp end 235 (FIG. 2) of the dilator 230 (FIG. 2) to secure the core 345 and ensure it is not proud relative to the surrounding native articular cartilage surface 350.

Although the above embodiments have been described in language that is specific to certain structures, elements, compositions, and methodological steps, it is to be understood that the technology defined in the appended claims is not necessarily limited to the specific structures, elements, compositions and/or steps described. Rather, the specific aspects and steps are described as forms of implementing the claimed technology. Since many embodiments of the technology can be practiced without departing from the spirit and scope of the invention, the invention resides in the claims hereinafter appended.

What is claimed is:

1. A defect sizer tool providing a measurement guide tool for selection of an appropriately sized pre-cut core for a cryopreserved osteochondral allograft (COCA) transplantation procedure with a corresponding instrument set, the defect sizer tool comprising:
a first end and a second end in opposition to one another, an elongated connector extending between the first end and the second end, and the elongated connector providing a handle portion configured to allow a user to grasp the handle portion between the first end and the second end; and
a first ring measurement guide disposed at the first end, the first ring measurement guide has a first given diameter, and the first ring measurement guide is configured to align with a cartilage defect to provide a visual indication for an estimated measurement of a diameter of the cartilage defect.

2. The defect sizer tool in accordance with claim 1, wherein the first given diameter of the first ring measurement guide is an outer diameter of the first ring measurement guide.

3. The defect sizer tool in accordance with claim 2, wherein the first ring measurement guide includes a second additional given diameter configured to align with a cartilage defect to provide a visual indication for an estimated measurement of a diameter of the cartilage defect, and the second additional given diameter is an inner diameter of the first ring measurement guide.

4. The defect sizer tool in accordance with claim 1, further comprising a second ring measurement guide disposed at the second end, the second ring measurement guide has a second given diameter, the first given diameter of the first second ring measurement guide and the second given diameter of the second ring measurement guide are unequal to one another so as to provide at least two measurement guides on the defect sizer tool, and the second ring measurement guide is configured to align with a cartilage defect to provide a visual indication for an estimated measurement of a diameter of the cartilage defect.

5. The defect sizer tool in accordance with claim 4, wherein the first given diameter of the first ring measurement guide is an outer diameter of the first ring measurement guide, the second given diameter of the second ring measurement guide is an outer diameter of the first ring measurement guide, and wherein the outer diameter of the first ring measurement guide and the outer diameter of the second ring measurement guide are unequal to one another so as to provide at least two measurement guides on the defect sizer tool.

6. The defect sizer tool in accordance with claim 5:
wherein the first ring measurement guide includes a second additional given diameter configured to align with a cartilage defect to provide a visual indication for an estimated measurement of a diameter of the cartilage defect, and the second additional given diameter of the first ring measurement guide is an inner diameter of the first ring measurement guide;
wherein the second ring measurement guide includes a second additional given diameter configured to align with a cartilage defect to provide a visual indication for an estimated measurement of a diameter of the cartilage defect, and the second additional given diameter of the second ring measurement guide is an inner diameter of the second ring measurement guide; and
wherein the outer diameter of the first ring measurement guide, the second additional given diameter of the first ring measurement guide, the outer diameter of the second ring measurement guide, and the second additional given diameter of the second ring measurement guide are each unequal to one another so as to provide at least four measurement guides on the defect sizer tool.

7. The defect sizer tool in accordance with claim 6:
wherein the outer diameter of the first ring measurement guide has a diameter of 16 mm;
wherein the second additional given diameter of the first ring measurement guide has a diameter of 11 mm;
wherein the outer diameter of the second ring measurement guide has a diameter of 25 mm;
wherein the second additional given diameter of the second ring measurement guide has a diameter of 20 mm; and
wherein the defect sizer tool has at least four measurement guides thereon.

8. An instrument kit for a cryopreserved osteochondral allograft (COCA) transplantation procedure, the instrument kit comprising:
a pin having a first end and a second end in opposition to one another, a given outer diameter, and the first end configured to penetrate subchondral bone;
a pin guide having a first end and a second end, the pin guide forming a passageway extending between the first end and the second end, the passageway configured to slidingly receive the pin, with the width of the passageway corresponding to the given outer diameter of the pin to allow sliding motion of the pin therein without deflection from a longitudinal axis extending between the first end and the second end of the pin; and
a reamer with an adjustable stop, the reamer having a first end and a second end, the reamer forming a passageway extending between the first and the second end, the passageway configured to slidingly receive the pin, with the width of the passageway corresponding to the given outer diameter of the pin to allow sliding motion of the pin therein without deflection from a longitudinal axis extending between the first end and the second end of the pin, a set of blades at the first end, and a rotational drive connector at the second end.

9. The instrument kit in accordance with claim 8, wherein the pin guide has a given length, the pin has a suitable length corresponding to the given length of the pin guide, the suitable length being longer than the given length of the pin guide to penetrate subchondral bone beyond the pin guide, and the suitable length being shorter than 150 percent of the given length of the pin guide.

10. The instrument kit in accordance with claim 8, wherein the pin guide has a pin guide window having a given diameter, and the pin guide window configured to center the passageway of the pin guide with respect to an osteochondral defect and encompass the osteochondral defect.

11. The instrument kit in accordance with claim 10, wherein the pin guide window is positionable to in a flush orientation with the osteochondral defect and provide the passageway of the pin guide in a perpendicular orientation with a surface of the cartilage.

11. The instrument kit in accordance with claim 8, wherein the adjustable stop of the reamer is configured to provide adjustments depths of 5 mm, 7.5 mm, 10 mm, 12.5 mm and 15 mm.

12. The instrument kit in accordance with claim 8, wherein the reamer has a blade construction with winged blades, and the blade construction is configured to score cartilage before cutting the subchondral bone so as to provide a cut in the subchondral bone with a clean profile at a surgical site.

13. The instrument kit in accordance with claim 12, wherein the reamer includes a reamer bit portion and scoring bit portion integral with one another, the reamer providing the scoring bit portion at a location to first score surrounding cartilage prior to the reamer bit portion reaming the subchondral bone to the desired depth.

14. The instrument kit in accordance with claim 8, wherein the adjustable stop of the reamer is configured to adjust the cutting depth of the reamer by depressing a button and sliding the reamer to a desired depth.

15. The instrument kit in accordance with claim 8, wherein the reamer is configured attach to a drill thereto and ream coaxially along the pin into the subchondral bone until the adjustable stop is flush with a cartilage surface so as to create a recipient socket.

16. The instrument kit in accordance with claim 8, further comprising a depth guide configured for measurement of an osteochondral allograft recipient socket.

17. The instrument kit in accordance with claim 16, wherein the depth guide includes a depth stop with a right angle 255 configured to allow a user to obtain a flush engagement adjacent to the reamer so obtain a highly precise measurement.

18. The instrument kit in accordance with claim 8, further comprising a dilator with tamp, and the tamp contoured with a concavity of a core to ensure contact around the entire surface during insertion of the core so as to ensures direct contact distributed across the core for minimizing damage and allowing for optimized insertion of the core.

19. The instrument kit in accordance with claim 8, wherein the dilator with tamp further includes suture grips configured to wrap suture around a distal portion of a graft and retain the graft to remain in selective attachment to the suture grips for selective removal from a patient until final placement of the graft.

20. A method for a cryopreserved osteochondral allograft (COCA) transplantation procedure, the method comprising:
providing an instrument kit for a cryopreserved osteochondral allograft (COCA) transplantation procedure, the instrument kit comprising:
a pin having a first end and a second end in opposition to one another, a given outer diameter, and the first end configured to penetrate subchondral bone;
a pin guide having a first end and a second end, the pin guide forming a passageway extending between the first end and the second end, the passageway configured to slidingly receive the pin, with the width of the passageway corresponding to the given outer diameter of the pin to allow sliding motion of the pin therein without deflection from a longitudinal axis extending between the first end and the second end of the pin; and
a reamer with an adjustable stop, the reamer having a first end and a second end, the reamer forming a passageway extending between the first and the second end, the passageway configured to slidingly receive the pin, with the width of the passageway corresponding to the given outer diameter of the pin to allow sliding motion of the pin therein without deflection from a longitudinal axis extending between the first end and the second end of the pin, a set of blades at the first end, and a rotational drive connector at the second end;
using the pin guide to place the pin in the subchondral bone; and
operating the reamer to first score surrounding cartilage prior to the reamer bit portion reaming the subchondral bone to the desired depth.
